(19) United States
(12) Patent Application Publication
(10) Pub. No.: US 2025/0199325 A1
(43) Pub. Date: Jun. 19, 2025

(54) FRAGRANCE DEVICE FOR VIRTUAL REALITY HEADSET

(71) Applicant: Sourabh Jain, San Francisco, CA (US)

(72) Inventor: Sourabh Jain, San Francisco, CA (US)

(21) Appl. No.: 19/071,989

(22) Filed: Mar. 6, 2025

Related U.S. Application Data
(63) Continuation-in-part of application No. 18/195,580, filed on May 10, 2023.
(60) Provisional application No. 63/329,669, filed on Apr. 11, 2022.

(51) Int. Cl. G02B 27/01; A61M 21/00

(52) U.S. Cl. CPC G02B 27/0176; A61M 21/00; A61M 2021/0016; A61M 2205/507

(57) ABSTRACT

The present disclosure provides a device for augmenting a virtual reality experience. The device includes an absorbent pad configured to absorb a fragrant liquid, a housing configured to hold the absorbent pad within while allowing fragrance of the pad to permeate through or escape out of the housing, and a coupling mechanism for detachably coupling the housing to an exterior surface of a virtual reality headset. The housing may be in the form of a locket with first and second sections coupled by a hinge and a latch mechanism for holding the locket closed. The coupling mechanism may comprise an adhesive patch for mounting to the virtual reality headset. The absorbent pad may be made of cotton felt, and the housing may include holes for applying fragrant liquid.

### CROSS REFERENCE TO RELATED APPLICATIONS

This application is a continuation in part of U.S. application Ser. No. 18/195,580, titled DEVICE FOR AUGMENTING A VIRTUAL REALITY EXPERIENCE filed May 10, 2023, and claims priority to U.S. Application No. 63/329,669, titled DEVICE FOR AUGMENTING A VIRTUAL REALITY EXPERIENCE, filed Apr. 11, 2022, which are hereby incorporated by reference in their entirety.

### FIELD OF INVENTION

The present disclosure relates to devices for enhancing virtual reality experiences, and more particularly to a detachable fragrance device for mounting on a virtual reality headset to provide olfactory stimulation during use.

### BACKGROUND

Virtual reality (VR) technology has become increasingly popular and accessible in recent years. VR headsets allow users to immerse themselves in computer-generated environments, providing visual and auditory stimulation to create the illusion of being present in a virtual world. These devices have found applications in gaming, education, training simulations, and various other fields.

While VR systems excel at engaging users' visual and auditory senses, they often lack stimulation of other sensory modalities like touch, taste, and smell. This limitation can reduce the overall level of immersion and realism in virtual experiences. Incorporating additional sensory feedback could potentially enhance the user's sense of presence and make virtual environments feel more lifelike and engaging.

Olfactory stimulation, in particular, has the potential to add another dimension to VR experiences. The human sense of smell is closely linked to memory and emotion, making it a powerful tool for creating immersive and memorable virtual environments. However, integrating scent into VR systems presents several challenges. Existing solutions for delivering fragrances in VR contexts tend to be complex, bulky, or limited in the range of scents they can produce.

There is interest in developing simple, compact, and versatile methods for introducing olfactory elements into VR experiences. Ideally, such solutions would be compatible with existing VR hardware, allow for easy customization of scents, and not interfere with the ergonomics or functionality of VR headsets. Addressing these considerations could lead to more immersive and multisensory virtual reality experiences for users across various applications.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a device for augmenting a virtual reality experience is provided. The device includes an absorbent pad configured to absorb a fragrant liquid. The device also includes a housing configured to hold the absorbent pad within while allowing fragrance of the pad to permeate through or escape out of the housing. The device further includes a coupling mechanism for detachably coupling the housing to an exterior surface of a virtual reality headset.

According to other aspects of the present disclosure, the device may include one or more of the following features. The absorbent pad may be made of cotton felt. The absorbent pad may be made of a material selected from the group consisting of wool, nylon, microfiber, felt, and any combination thereof. The housing may be in the form of a locket having first and second sections coupled by a hinge and may comprise a latch mechanism for holding the locket closed. The housing may be in the form of a clip having first and second sections held together by a spring-like bearing. The housing may be made of a material selected from the group consisting of wood, plastic, metal, metal alloy, cork, leather, and any combination thereof.

The coupling mechanism may comprise a first Velcro piece attached to the housing and a corresponding second Velcro piece having a flat rear surface coated in adhesive for mounting it to the exterior surface of a virtual reality headset. The coupling mechanism may be selected from the group consisting of adhesive pad, buttons, snaps, laces, hook-and-eye fasteners, buckles, twist-ties, magnets, slide-flat hooks, safety pins, rubber bands, adhesive, tape, and any combinations thereof. An exterior surface of the housing may be provided with an aesthetic design, shape, or engraving.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates an exploded view of a fragrance device, according to aspects of the present disclosure.

FIG. 2 illustrates an isometric view of the fragrance device of FIG. 1 in an assembled state, according to an embodiment.

FIG. 3 illustrates an isometric view of a virtual reality headset with a mounting patch, according to aspects of the present disclosure.

FIG. 4 illustrates an isometric view of the fragrance device of FIG. 1 mounted on the virtual reality headset of FIG. 3, according to an embodiment.

FIG. 5 illustrates an isometric view of the underside of a virtual reality headset with mounting patches, according to aspects of the present disclosure.

FIG. 6 illustrates an isometric view of a virtual reality headset with fragrance devices installed, according to an embodiment.

FIG. 7 illustrates a bottom view of a virtual reality headset with fragrance devices installed, according to an embodiment.

FIG. 8 illustrates a bottom view of a virtual reality headset with fragrance devices installed, according to an alternate embodiment.

FIG. 9 illustrates a bottom view of a virtual reality headset with fragrance devices installed, according to an alternate embodiment.

FIG. 10 shows a view from inside the fragrance device wherein the at least one hole configured as an iris.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to a device and method for augmenting a virtual reality experience by providing an olfactory element. The device may include a housing configured to hold an absorbent pad, which may be infused with a fragrant liquid. The housing may be designed to allow the fragrance to permeate or escape, while also providing a means for detachably coupling the device to an exterior surface of a virtual reality headset.

In some cases, the device may enhance the immersive quality of virtual reality experiences by introducing relevant scents to complement visual and auditory stimuli. The device may be compact and simple in design, facilitating easy attachment to and removal from virtual reality headsets. Additionally, the device may allow for convenient switching between different fragrances, providing flexibility in adapting to various virtual environments or user preferences.

The absorbent pad within the device may be composed of materials capable of effectively retaining and releasing fragrant liquids. The housing may be constructed from a variety of materials and may incorporate features to securely contain the absorbent pad while allowing for the controlled release of fragrance.

A coupling mechanism may be included as part of the device, enabling users to attach and detach the fragrance device from a virtual reality headset. This mechanism may be designed to provide secure attachment without interfering with the normal operation or comfort of the headset.

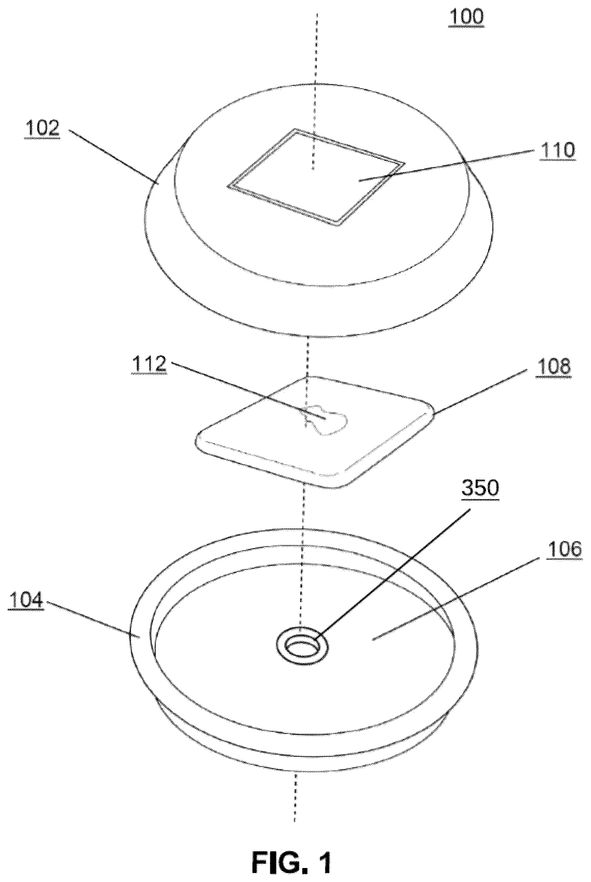

As shown in FIG. 1 the fragrance device 100 may include a housing structure configured to contain and protect an absorbent pad while allowing fragrance to permeate or escape. In some cases, the housing structure may comprise a first housing section 102 and a second housing section 104, which together define an interior volume 106 when assembled.

The housing structure of the fragrance device 100 may take various forms. In some cases, the housing structure may be in the form of a locket with the first housing section 102 and the second housing section 104 coupled by a hinge. The housing structure may include a latch mechanism for holding the locket closed. In other cases, the housing structure may be in the form of a clip with the first housing section 102 and the second housing section 104 held together by a spring-like bearing.

The material forming the housing structure of the fragrance device 100 may vary. In some cases, the housing structure may be constructed from wood, plastic, metal, metal alloy, cork, leather, or any combination thereof. The choice of material may depend on factors such as durability, weight, cost, or aesthetic preferences.

In some cases, an exterior surface of the housing structure may be provided with an aesthetic design, shape, or engraving. This may allow for customization or branding of the fragrance device 100.

The first housing section 102 may contain at least one hole 350 to allow drops of fragrant liquid through to the absorbent pads within the interior volume 106. These holes may facilitate refilling of the fragrance device 100, potentially reducing time and cost associated with replenishing the fragrance. The at least one hole 350 may be of various shapes, provided they are large enough to allow fragrant liquids to pass through to the absorbent pads.

Alternatively, at least one hole 350 may be an iris that enables the user to adjust the opening through which the fragrance can escape from the fragrance device 100 thereby allowing the user to control the amount of fragrance dispensed from the fragrance device 100,

The configuration of the housing structure may allow for easy access to the interior volume 106 for replacing or refilling the absorbent pad. In some cases, the first housing section 102 and the second housing section 104 may be separable to provide access to the interior volume 106. The housing structure may be designed to securely close, preventing unintended leakage of fragrant liquid while allowing controlled release of fragrance.

The fragrance device 100 may include an absorbent pad 108 configured to absorb and retain fragrant liquids. In some cases, the absorbent pad 108 may be composed of cotton felt. However, the absorbent pad 108 may also be made from a variety of other materials. For example, the absorbent pad 108 may be composed of wool, nylon, microfiber, felt, or any combination thereof. The choice of material for the absorbent pad 108 may depend on factors such as absorption capacity, retention properties, and compatibility with different types of fragrant liquids.

The absorbent pad 108 may be designed to fit within the interior volume 106 formed by the first housing section 102 and the second housing section 104 of the fragrance device 100. The size and shape of the absorbent pad 108 may be tailored to maximize the use of the available space within the interior volume 106, potentially increasing the amount of fragrant liquid that can be held.

In some cases, the absorbent pad 108 may contain a fragrant liquid patch 112. The fragrant liquid patch 112 may represent an area of the absorbent pad 108 where fragrant liquid has been absorbed. The size and distribution of the fragrant liquid patch 112 may vary depending on the amount and type of fragrant liquid applied, as well as the absorption properties of the absorbent pad 108.

The first housing section 102 of the fragrance device 100 may include at least one hole 350 to facilitate the application of fragrant liquid to the absorbent pad 108. These holes may be designed to allow drops of fragrant liquid to pass through to the absorbent pad 108 contained within the interior volume 106. The presence of these holes may simplify the process of refilling or replenishing the fragrance in the device, potentially reducing the time and effort required for maintenance.

The at least one hole 350 in the first housing section 102 may vary in shape and size. In some cases, the holes may be circular, while in other cases, they may be oval, square, or any other shape that allows for the effective passage of fragrant liquid. The key consideration for the design of these holes may be ensuring that they are large enough to permit the easy application of fragrant liquid to the absorbent pad 108.

The configuration of the absorbent pad 108 and the holes in the first housing section 102 may allow for the controlled release of fragrance from the fragrance device 100. As the fragrant liquid evaporates from the absorbent pad 108, the resulting scent may permeate through the holes or other openings in the housing of the fragrance device 100, providing an olfactory element to enhance the virtual reality experience.

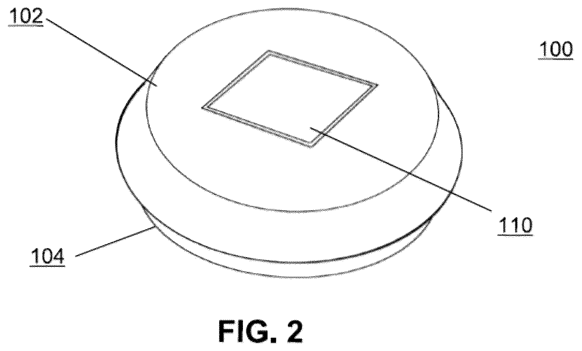

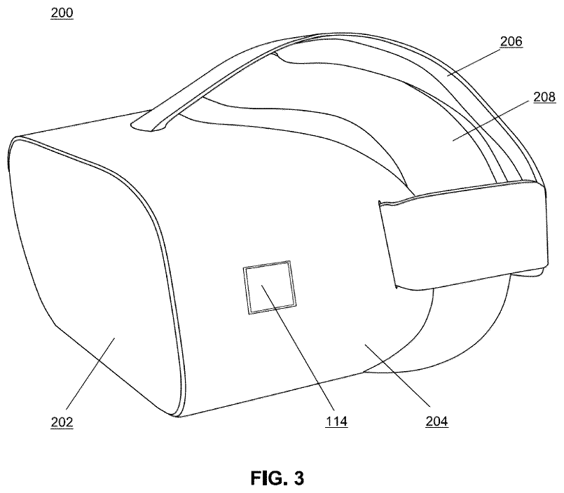

As shown in FIG. 2 the fragrance device 100 may include a coupling mechanism for attaching the device to a virtual reality headset 200. In some cases, the coupling mechanism may comprise a coupling patch 110 attached to the exterior of the fragrance device 100. The coupling patch 110 may be designed to interact with a corresponding mounting patch 114 on the virtual reality headset 200 as shown in FIG. 3.

FIG. 3 illustrates an isometric view of a virtual reality headset 200 with a mounting patch 114 attached to the side surface 204. The mounting patch 114 may provide a surface for attaching the fragrance device 100 to the virtual reality headset 200.

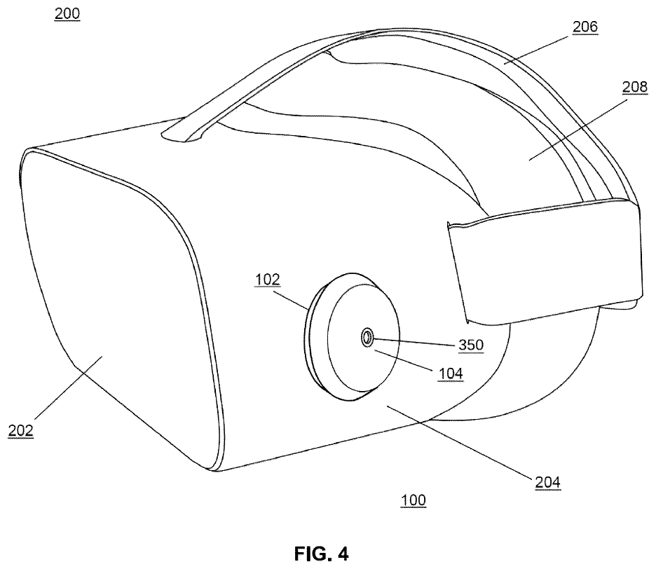

FIG. 4 shows the fragrance device 100 mounted on the side surface 204 of the virtual reality headset 200. The coupling patch 110 on the fragrance device 100 may interact with the mounting patch 114 on the virtual reality headset 200 to secure the fragrance device 100 in place.

In some cases, the coupling patch 110 and the mounting patch 114 may utilize hook-and-loop fasteners, such as Velcro or by using an adhesive patch. However, the coupling mechanism may employ various alternative attachment methods. For example, the coupling mechanism may include an adhesive patch, buttons, snaps, laces, hook-and-eye fasteners, buckles, twist-ties, magnets, slide-flat hooks, safety pins, rubber bands, adhesive, tape, or combinations thereof.

The choice of attachment method for the coupling mechanism may depend on factors such as the desired ease of attachment and detachment, the weight of the fragrance device 100, and the surface characteristics of the virtual reality headset 200. For instance, magnetic attachments may provide quick and easy coupling and decoupling, while adhesive attachments may offer a more permanent solution.

In some cases, the coupling mechanism may be designed to be easily removable, allowing users to quickly attach or detach the fragrance device 100 from the virtual reality headset 200. This feature may facilitate the swapping of different fragrance devices 100 or the removal of the device when not in use.

The positioning of the coupling patch 110 on the fragrance device 100 may be optimized to ensure secure attachment to the virtual reality headset 200 without interfering with the operation of the first housing section 102 and the second housing section 104. Similarly, the location of the mounting patch 114 on the virtual reality headset 200 may be chosen to allow convenient access to the fragrance device 100 while maintaining the overall balance and comfort of the headset.

The fragrance device 100 may be integrated with various parts of the virtual reality headset 200 to enhance the user's experience. FIG. 3 illustrates an isometric view of the virtual reality headset 200, which includes a front housing 202, a rear housing 208, and a headset strap 206 connecting the front housing 202 and the rear housing 208. The side surface 204 of the virtual reality headset 200 may provide one potential mounting location for the fragrance device 100.

FIG. 4 demonstrates how the fragrance device 100 may be mounted on the side surface 204 of the virtual reality headset 200. In this configuration, the fragrance device 100 may be positioned near the user's face, allowing for effective delivery of fragrances via at least one hole 350 during use of the virtual reality headset 200. The placement on the side surface 204 may not interfere with the user's field of view or the operation of the headset strap 206.

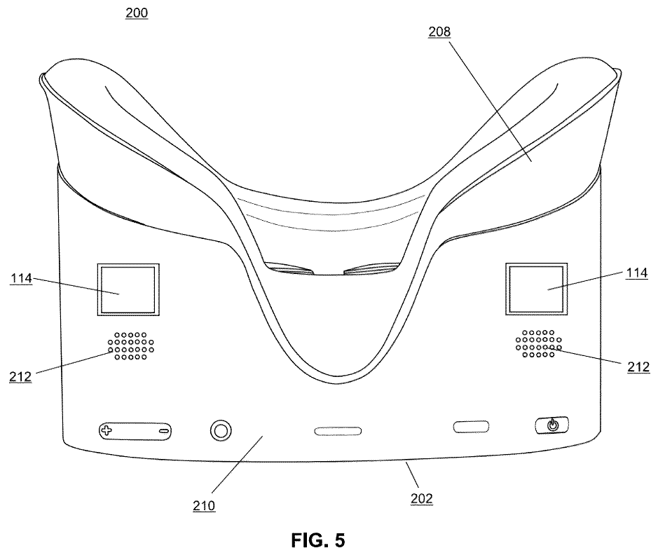

In some cases, the fragrance device 100 may be mounted on alternative locations of the virtual reality headset 200. FIG. 5 illustrates an isometric view of the underside of the virtual reality headset 200, revealing a bottom surface 210. The bottom surface 210 may include speaker arrays 212, which may be positioned near the user's ears during operation of the virtual reality headset 200. the mounting patch 114 on the virtual reality headset 200 supports attachment of fragrance device 100 to the virtual reality headset 200.

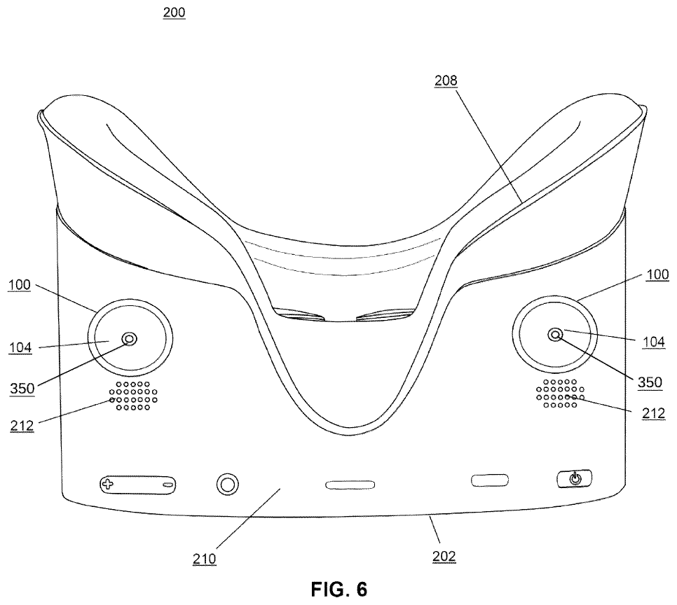

FIG. 6 shows how the fragrance device 100 may be mounted on the bottom surface 210 of the virtual reality headset 200. In this configuration, the fragrance device 100 may be positioned near the speaker arrays 212, which may place the fragrance device 100 in close proximity to the user's nose. This placement may allow for more direct delivery of fragrances to the user's olfactory senses.

The mounting of the fragrance device 100 on different parts of the virtual reality headset 200 may offer various benefits. Mounting on the side surface 204, as shown in FIG. 4, may provide easy access for users to replace or refill the fragrance device 100. In contrast, mounting on the bottom surface 210, as illustrated in FIG. 6, may position the fragrance device 100 closer to the user's nose, potentially enhancing the intensity of the fragrance experience.

In some cases, multiple fragrance devices 100 may be mounted on the virtual reality headset 200 simultaneously. For example, one fragrance device 100 may be mounted on each side surface 204, or two fragrance devices 100 may be mounted symmetrically on the bottom surface 210, as shown in FIG. 6. This configuration may allow for the delivery of multiple fragrances or may enhance the overall intensity of a single fragrance.

The integration of the fragrance device 100 with the virtual reality headset 200 may be achieved through the use of the coupling patch 110 on the fragrance device 100 and the corresponding mounting patch 114 on the virtual reality headset 200. These patches may allow for secure attachment while also enabling easy removal or repositioning of the fragrance device 100 as needed.

The fragrance device 100 may operate in conjunction with the virtual reality headset 200 to enhance the user's immersive experience. In some cases, the process of using the fragrance device 100 may involve several steps, from initial setup to ongoing operation.

The setup process may begin with the attachment of the fragrance device 100 to the virtual reality headset 200. As shown in FIG. 4, the fragrance device 100 may be mounted on the side surface 204 of the virtual reality headset 200. In some cases, the coupling patch 110 on the fragrance device 100 may interact with the mounting patch 114 on the virtual reality headset 200 to secure the fragrance device 100 in place.

Alternatively, as illustrated in FIG. 6, the fragrance device 100 may be attached to the bottom surface 210 of the virtual reality headset 200. This configuration may position the fragrance device 100 closer to the user's nose, potentially enhancing the intensity of the fragrance experience.

Once the fragrance device 100 is securely attached to the virtual reality headset 200, the first housing section 102 may be opened to access the interior volume 106. The absorbent pad 108 may then be placed within the interior volume 106. In some cases, the absorbent pad 108 may already contain a fragrant liquid patch 112. If not, fragrant liquid may be applied to the absorbent pad 108 at this stage.

The first housing section 102 may contain at least one hole 350 to facilitate the application of fragrant liquid to the absorbent pad 108. These holes may allow drops of fragrant liquid to pass through to the absorbent pad 108 contained within the interior volume 106. The shape of the at least one hole 350 may vary, provided they are large enough to permit the passage of fragrant liquid and the at least one hole 350 shape can be round, square, elliptical, rectangular or any shape suitable for dispersing the fragrance.

After the absorbent pad 108 is in place and infused with fragrant liquid, the first housing section 102 and the second housing section 104 may be closed to secure the absorbent pad 108 within the fragrance device 100.

During operation of the virtual reality headset 200, the fragrant liquid in the absorbent pad 108 may gradually evaporate. The resulting scent may permeate through the holes or other openings in the housing of the fragrance device 100, providing an olfactory element to complement the visual and auditory stimuli of the virtual reality experience.

The fragrance device 100 may be refilled or replenished as needed. The holes in the first housing section 102 may allow for the addition of fragrant liquid without the need to open the fragrance device 100 fully. This feature may simplify the maintenance process, potentially reducing the time and effort required to keep the fragrance device 100 operational.

In some cases, multiple fragrance devices 100 may be used simultaneously with a single virtual reality headset 200. For example, as shown in FIG. 6, two fragrance devices 100 may be mounted on the bottom surface 210 of the virtual reality headset 200. This configuration may allow for the delivery of multiple fragrances or may enhance the overall intensity of a single fragrance.

The fragrance device 100 may be easily detached from the virtual reality headset 200 when not in use or when a different fragrance is desired. The coupling mechanism may allow for quick removal and reattachment, facilitating the swapping of different fragrance devices 100 or fragrances to suit various virtual environments or user preferences.

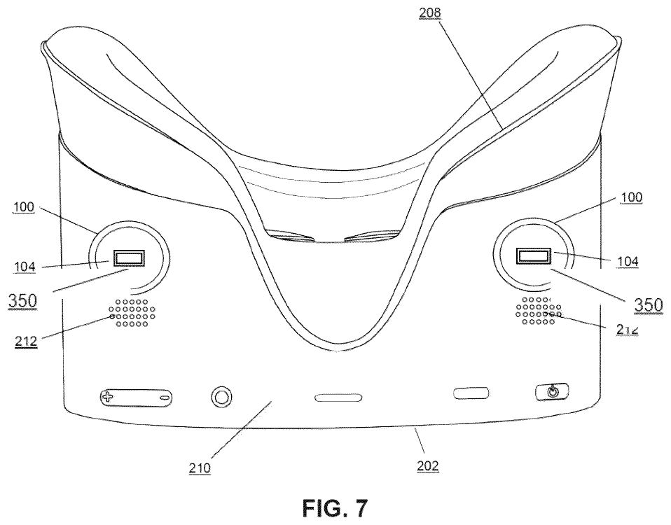

FIG. 7 illustrates a bottom view of a virtual reality headset with fragrance devices installed, according to an embodiment. FIG. 7 shows how the fragrance device 100 may be mounted on the bottom surface 210 of the virtual reality headset 200. In this configuration, the fragrance device 100 may be positioned near the speaker arrays 212, which may place the fragrance device 100 in close proximity to the user's nose. This placement may allow for more direct delivery of fragrances to the user's olfactory senses.

The mounting of the fragrance device 100 on different parts of the virtual reality headset 200 may offer various benefits. Mounting on the side surface 204, as shown in FIG. 4, may provide easy access for users to replace or refill the fragrance device 100. In contrast, mounting on the bottom surface 210, as illustrated in FIG. 7, may position the fragrance device 100 closer to the user's nose, potentially enhancing the intensity of the fragrance experience.

The first housing section 102 may contain at least one hole 350 to facilitate the application of fragrant liquid to the absorbent pad 108. These holes may allow drops of fragrant liquid to pass through to the absorbent pad 108 contained within the interior volume 106. The shape of the at least one hole 350 may vary, provided they are large enough to permit the passage of fragrant liquid and the at least one hole 350 shape can be round, square, elliptical, hexagon, triangular, rectangular or any shape suitable for dispersing the fragrance.

Alternatively, at least one hole 350 may be an iris that enables the user to adjust the opening through which the fragrance can escape from the fragrance device 100 thereby allowing the user to control the amount of fragrance dispensed from the fragrance device 100,

In some cases, multiple fragrance devices 100 may be mounted on the virtual reality headset 200 simultaneously. For example, one fragrance device 100 may be mounted on each side surface 204, or two fragrance devices 100 may be mounted symmetrically on the bottom surface 210, as shown in FIG. 7 This configuration may allow for the delivery of multiple fragrances or may enhance the overall intensity of a single fragrance.

The integration of the fragrance device 100 with the virtual reality headset 200 may be achieved through the use of the coupling patch 110 on the fragrance device 100 and the corresponding mounting patch 114 on the virtual reality headset 200. These patches may allow for secure attachment while also enabling easy removal or repositioning of the fragrance device 100 as needed.

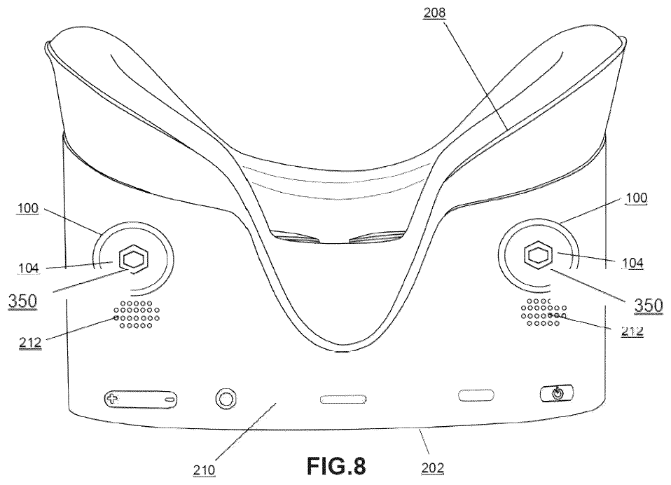

FIG. 8 illustrates a bottom view of a virtual reality headset with fragrance devices installed, according to an alternate embodiment. FIG. 8 illustrates a bottom view of a virtual reality headset with fragrance devices installed, according to an embodiment. FIG. 8 shows how the fragrance device 100 may be mounted on the bottom surface 210 of the virtual reality headset 200. In this configuration, the fragrance device 100 may be positioned near the speaker arrays 212, which may place the fragrance device 100 in close proximity to the user's nose. This placement may allow for more direct delivery of fragrances to the user's olfactory senses.

The mounting of the fragrance device 100 on different parts of the virtual reality headset 200 may offer various benefits. Mounting on the side surface 204, as shown in FIG. 4, may provide easy access for users to replace or refill the fragrance device 100. In contrast, mounting on the bottom surface 210, as illustrated in FIG. 8, may position the fragrance device 100 closer to the user's nose, potentially enhancing the intensity of the fragrance experience.

The first housing section 102 may contain at least one hole 350 to facilitate the application of fragrant liquid to the absorbent pad 108. These holes may allow drops of fragrant liquid to pass through to the absorbent pad 108 contained within the interior volume 106. The shape of the at least one hole 350 may vary, provided they are large enough to permit the passage of fragrant liquid and the and the at least one hole 350 shape can be round, square, elliptical, hexagon, triangular, rectangular or any shape suitable for dispersing the fragrance.

In some cases, multiple fragrance devices 100 may be mounted on the virtual reality headset 200 simultaneously. For example, one fragrance device 100 may be mounted on each side surface 204, or two fragrance devices 100 may be mounted symmetrically on the bottom surface 210, as shown in FIG. 8. This configuration may allow for the delivery of multiple fragrances or may enhance the overall intensity of a single fragrance.

The integration of the fragrance device 100 with the virtual reality headset 200 may be achieved through the use of the coupling patch 110 on the fragrance device 100 and the corresponding mounting patch 114 on the virtual reality headset 200. These patches may allow for secure attachment while also enabling easy removal or repositioning of the fragrance device 100 as needed.

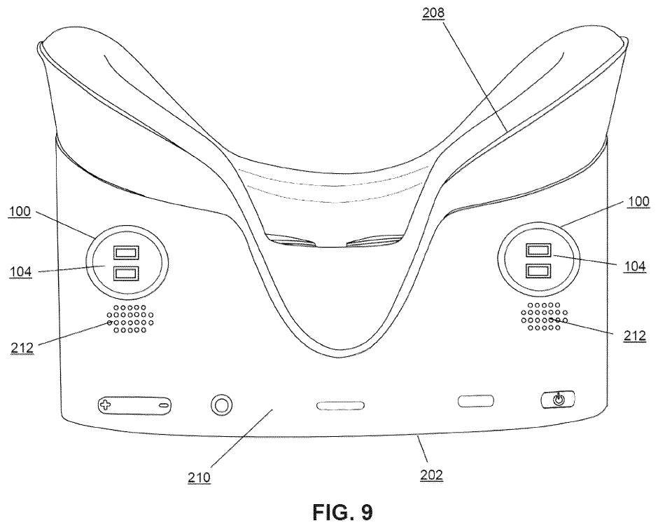

FIG. 9 illustrates a bottom view of a virtual reality headset with fragrance devices installed, according to an alternate embodiment. FIG. 9 illustrates a bottom view of a virtual reality headset with fragrance devices installed, according to an alternate embodiment. FIG. 9 illustrates a bottom view of a virtual reality headset with fragrance devices installed, according to an embodiment. FIG. 9 shows how the fragrance device 100 may be mounted on the bottom surface 210 of the virtual reality headset 200. In this configuration, the fragrance device 100 may be positioned near the speaker arrays 212, which may place the fragrance device 100 in close proximity to the user's nose. This placement may allow for more direct delivery of fragrances to the user's olfactory senses.

The mounting of the fragrance device 100 on different parts of the virtual reality headset 200 may offer various benefits. Mounting on the side surface 204, as shown in FIG. 4, may provide easy access for users to replace or refill the fragrance device 100. In contrast, mounting on the bottom surface 210, as illustrated in FIG. 8, may position the fragrance device 100 closer to the user's nose, potentially enhancing the intensity of the fragrance experience.

The first housing section 102 may contain at least one hole 350 to facilitate the application of fragrant liquid to the absorbent pad 108. These holes may allow drops of fragrant liquid to pass through to the absorbent pad 108 contained within the interior volume 106. The shape of the at least one hole 350 may vary, provided they are large enough to permit the passage of fragrant liquid and the and the at least one hole 350 shape can be round, square, elliptical, hexagon, triangular, rectangular or any shape suitable for dispersing the fragrance.

Alternatively, at least one hole 350 may be an iris that enables the user to adjust the opening through which the fragrance can escape from the fragrance device 100 thereby allowing the user to control the amount of fragrance dispensed from the fragrance device 100,

In some cases, multiple fragrance devices 100 may be mounted on the virtual reality headset 200 simultaneously. For example, one fragrance device 100 may be mounted on each side surface 204, or two fragrance devices 100 may be mounted symmetrically on the bottom surface 210, as shown in FIG. 8. This configuration may allow for the delivery of multiple fragrances or may enhance the overall intensity of a single fragrance.

The integration of the fragrance device 100 with the virtual reality headset 200 may be achieved through the use of the coupling patch 110 on the fragrance device 100 and the corresponding mounting patch 114 on the virtual reality headset 200. These patches may allow for secure attachment while also enabling easy removal or repositioning of the fragrance device 100 as needed.

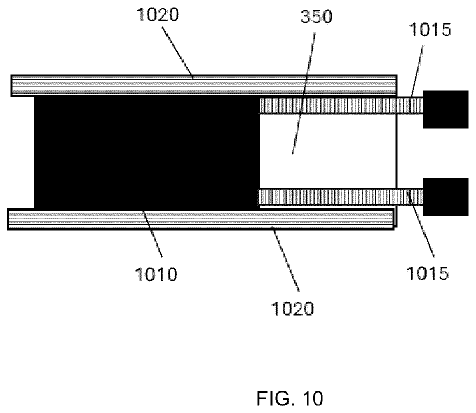

FIG. 10 shows a view from inside the fragrance device 100 wherein the at least one hole 350 configured as an iris. As shown in FIG. 10 the iris is a door 1010 that has at least one Nitinol metal pullers 1015 that changes length when electricity is applied. Typical metals are Nitinol (Nickel-Titanium alloy), which is often referred to as a “shape-memory alloy” and is commonly used in applications where a material needs to change shape based on an electric current, like in “muscle wire” technology. The door runs in slides 1020. Alternatively, the iris could be adjusted manually by the user, or the iris could use springs to open and close the iris shutter and the motion of the springs is controlled by the user.

When the iris is controlled electrically the fragrance device 100 could have a controller such as a Bluetooth microcontroller that controls the at least one metal pullers 1015 by applying a current to the at least one metal pullers 1015. The Bluetooth microcontroller could be controlled by a remote-control devo-ice that is operated by the user. If the iris is controlled by a Bluetooth microcontroller then the fragrance device 100 would have the appropriate battery and controls need to power the Bluetooth microcontroller.

A number of implementations have been described. Nevertheless, it will be understood that various modifications may be made without departing from the spirit and scope of the disclosure. Accordingly, other implementations are within the scope of the following claims.

1. A device for augmenting a virtual reality experience, comprising:
an absorbent pad configured to absorb a fragrant liquid;
a housing comprising a first housing section and a second housing section and said housing is configured to hold the absorbent pad within said housing and said pad is capable of absorbing a liquid fragrance and said pad allowing said fragrance absorbed into the pad to permeate through or escape out of the housing through at least one hole in said second housing section and said at least one hole is in communication with said pad so that said liquid fragrance can be applied to said pad using said at least on hole by a user of said device; and
a coupling mechanism for detachably coupling the housing to an exterior surface of a virtual reality headset.

2. The device of claim 1, wherein the housing is in the form of a locket having first and second sections coupled by a hinge, the housing further comprising a latch mechanism for holding the locket closed.

3. The device of claim 2, wherein the housing comprises one or more of wood, plastic, metal, metal alloy, cork and leather.

4. The device of claim 1, wherein the coupling mechanism comprises an adhesive for mounting it to the exterior surface of the virtual reality headset.

5. The device of claim 1, wherein an exterior surface of the housing is provided with an aesthetic design, shape, or engraving.

6. The device of claim 1, wherein the absorbent pad comprises a piece of cotton felt.

7. The device of claim 6, wherein the housing includes at least one hole configured to allow drops of fragrant liquid to pass through to the absorbent pad within the housing.

8. A method for augmenting a virtual reality experience, comprising:
placing an absorbent pad configured to absorb a liquid fragrance into a housing, the housing configured to hold the absorbent pad while allowing fragrance of the pad to permeate through or escape out of the housing through at least one hole;
applying a fragrant liquid to the absorbent pad though said at least one hole; and
detachably coupling the housing to an exterior surface of a virtual reality headset using a coupling mechanism.

9. The method of claim 8, wherein the housing is in the form of a locket having first and second sections coupled by a hinge, the method further comprising closing the locket after placing the absorbent pad within the housing.

10. The method of claim 9, wherein the housing further comprises a latch mechanism, the method further comprising engaging the latch mechanism to hold the locket closed.

11. The method of claim 8, wherein applying the fragrant liquid to the absorbent pad comprises dropping the fragrant liquid through at least one hole in the housing configured to allow the fragrant liquid to pass through to the absorbent pad.

12. The method of claim 11, wherein the absorbent pad comprises a piece of cotton felt.

13. The method of claim 8, wherein detachably coupling the housing to the exterior surface of the virtual reality headset comprises attaching an adhesive patch to the exterior surface of the virtual reality headset.

14. The method of claim 13, further comprising removing the housing from the virtual reality headset and attaching a second housing containing a different fragrant liquid to provide a different scent during the virtual reality experience.

15. A virtual reality system, comprising:
a virtual reality headset; and
a fragrance device detachably coupled to an exterior surface of the virtual reality headset, the fragrance device comprising:
a housing comprising a first housing section and a second housing section and said housing is configured to hold the absorbent pad within said housing and said pad is capable of absorbing a liquid fragrance and said pad allowing said fragrance absorbed into the pad to permeate through or escape out of the housing through at least one hole in said second housing section and said at least one hole is in communication with said pad so that said liquid fragrance can be applied to said pad using said at least on hole by a user of said device;
a coupling mechanism for detachably coupling the housing to the exterior surface of the virtual reality headset:
the at least one hole in said second housing section having an iris and said iris is controlled by at least one Nitinol metal pullers; and
said Nitinol metal pullers controlled by a microcontroller.

16. The virtual reality system of claim 15, wherein the housing is in the form of a locket having first and second sections coupled by a hinge, the housing further comprising a latch mechanism for holding the locket closed.

17. The virtual reality system of claim 16, wherein the housing comprises one or more of wood, plastic, metal, metal alloy, cork and leather.

18. The virtual reality system of claim 15, wherein the coupling mechanism comprises an adhesive patch for mounting it to the exterior surface of the virtual reality headset.

19. The virtual reality system of claim 15, wherein the absorbent pad comprises a piece of cotton felt.

20. The virtual reality system of claim 19, wherein the housing includes at least one hole configured to allow drops of fragrant liquid to pass through to the absorbent pad within the housing.
